SEQUENCE LISTING (12 sequences)

SEQ ID NO: 1 - protein, 14 aa, Bombina bombina
QQRLGNQWAVGHLM

SEQ ID NO: 2 - protein, 9 aa, Artificial Sequence
  Analogs of C-terminal bombesin fragments
XQWAVXHXX

SEQ ID NO: 3 - protein, 8 aa, Artificial sequence
  Preferred peptide sequence # 1
QWAVGHLX

SEQ ID NO: 4 - protein, 8 aa, Artificial sequence
  Preferred peptide sequence # 2
QWAVGHXX

SEQ ID NO: 5 - protein, 9 aa, Artificial sequence
  Preferred peptide sequence # 3
QWAVGHXXE

SEQ ID NO: 6 - protein, 8 aa, Artificial sequence
  Preferred peptide sequence # 4
QWAVGHXL

SEQ ID NO: 7 - protein, 8 aa, Artificial sequence
  Preferred peptide sequence # 5
QWAVXHXL

SEQ ID NO: 8 - protein, 9 aa, Artificial sequence
  Preferred peptide sequence # 6
XQWAVGHXL

SEQ ID NO: 9 - protein, 9 aa, Artificial sequence
  Preferred peptide sequence # 7
XQWAVGHXX

SEQ ID NO: 10 - protein, 9 aa, Artificial sequence
  Preferred peptide sequence # 8
XQWAVXHXL

SEQ ID NO: 11 - protein, 8 aa, Artificial sequence
  Preferred peptide sequence # 9
QWAVGHLM

SEQ ID NO: 12 - protein, 9 aa, Artificial sequence
  Preferred peptide sequence # 10
XQWAVXHLX

(19) United States
(12) Patent Application Publication
(10) Pub. No.: US 2014/0234211 A1
(43) Pub. Date: Aug. 21, 2014

(54) SUPRAMOLECULAR AGGREGATES CONTAINING AMPHIPHILIC MONOMERS, CHELATING AGENTS AND PEPTIDES FOR USE FOR DRUG DELIVERY AND AS CONTRAST AGENTS

(71) Applicant: INVECTORS S.r.l., NAPOLI (IT)

(72) Inventors: Antonella Accardo, Torre Del Greco (IT); Diego Tesauro, Napoli (IT); Giancarlo Morelli, Napoli (IT); Carlo Pedone, Napoli (IT); Giuseppe De Rosa, Napoli (IT); Giuseppina Salzano, Casavatore (IT)

(21) Appl. No.: 14/348,046

(22) PCT Filed: Sep. 28, 2012
(86) PCT No.: PCT/IB2012/055183

§ 371 Date: Mar. 27, 2014

(30) Foreign Application Priority Data
Sep. 28, 2011 (IT) TO2011A000864

(51) Int. Cl. A61K 9/127; A61K 51/12; A61K 31/704; A61K 47/48

(52) U.S. Cl. CPC A61K 9/127; A61K 47/48215; A61K 51/1234; A61K 31/704
USPC 424/1.21; 514/19.7; 424/450

(57) ABSTRACT

Delivery systems based on liposomes functionalized with peptides and chelating agents, for therapy and imaging by selective targeting of tumour cells expressing the receptors GRP, BB1, BB2, BB3 e BB4 and any other receptor which recognizes the bombesin peptide or analogues thereof are described. In particular, the liposomes contain within them cytotoxic drugs, such as for example doxorubicin, for target-selective antitumour therapy, and, through the presence of the chelating agent, can contain radioactive or paramagnetic ions for the real time visualization of the tumour cells. The liposomes described in this invention thus act as a selective delivery system for drugs and/or contrast agents onto tumour cells expressing the receptors for the class of known peptides such as bombesin (endogenous sequence, analogous or peptidomimetic peptides, agonists or non agonists).

The present invention relates to the preparation and use of supramolecular aggregates, such as for example liposomes, formulated with amphiphilic monomers functionalized with chelating agents and with peptides analogous to bombesin peptide (endogenous sequence, analogous or peptidomimetic peptides, agonists or non-agonists). The liposomes, loaded with cytotoxic drugs, such as for example doxorubicin, act as selective delivery systems for anti-tumour drugs and it is possible to visualize their biodistribution in real time using techniques from nuclear medicine (SPECT, PET) and magnetic resonance (MRI) through the presence of contrast media, such as radioactive or paramagnetic ions coordinated to the chelating agent.

The liposomes described in this invention therefore act as selective delivery systems for drugs and/or contrast agents onto tumour cells expressing receptors for the type of known peptides such as bombesin or analogues thereof, whether agonists or non-agonists. The target tumour cells therefore exhibit overexpression of the GRP, BB1, BB2, BB3, BB4 receptors or other bombesin receptors and are characteristic of some human tumours such as for example ovarian tumours, prostate tumours and tumours of the breast.

### STATE OF THE ART

Supramolecular systems such as micelles and liposomes are currently used for a variety of applications, both of diagnostic and therapeutic nature, such as for example gene delivery, formulation of vaccines and of new anti-tumour drugs, photodynamic therapy and the preparation of contrast media.

The majority of therapeutic agents are of limited applicability because of unfavourable pharmacokinetic properties, insufficient release of the drug into the tumour or at metastatic sites, and high organ toxicity. One method for reducing the toxicity associated with the drug and increasing its therapeutic index is the reformulation of the drug into liposomes. The advantages associated with a liposomal reformulation of the drug are: better therapeutic efficacy and better stability, biocompatibility and biodegradability. Liposomal systems can effect the release of the drug in a controlled way. The administration of an encapsulated drug limits the risk of creating fluctuations in plasma concentrations, furthermore it avoids the need for repeated administrations and allows the effective concentration to be maintained for a greater period of time. Currently a variety of drugs are available commercially in liposomal formulations (e.g. Abelcet®, AmBisome®, Pevaryl® lipogel, Epaxal Berna®, DauXonome®, Caelyx®, Myocet®, Visudyne®).

In particular, Myocet® and Doxil/Caelix® (Doxil is the trade name in the USA; Caelix in Europe) represent the two liposomal formulations of doxorubicin currently on the market, and both are used for the treatment of Kaposi's sarcoma and in ovarian and pulmonary tumours that are resistant to other chemotherapeutic agents. In contrast to Myocet, Doxil presents fragments of PEG (polyethylene glycol) on the outer surface of the aggregates. The PEG prevents the macrophages recognizing the liposomal drug as non-self, increasing its half-life. Other examples of liposomal medications in the experimental phase as chemotherapeutic agents are Allovectin-7 and Aroplatin. Allovectin is used as a chemotherapeutic agent for the treatment of phase III or IV metastatic malignant melanoma and is made up of a plasmid/lipid complex containing sequences of DNA encoding HLAB7-SS2 microglobulin, which in combination make up the major histo-compatibility complex MHC-I, provoking an immune response towards the tumour cells.

Aroplatin, on the other hand, contains platinum as an active ingredient and is currently under evaluation in clinical studies for the treatment of solid tumours and B cell lymphoma. This chemotherapy agent exerts its action by releasing platinum at the tumour cells, thus limiting the toxic effects of the latter in the kidneys and nervous system.

One strategic approach for selectively reaching the tumour cells is selective drug delivery, which can be effected by anchoring suitable bioactive molecules to the supramolecular aggregate such as antibodies, small molecules of organic nature or peptides. Many studies on immunoliposomes, made up of functionalized aggregates with fragments of monoclonal antibodies (mAb), have been reported in the literature. The antibodies recognize the antigens expressed onto the tumour-associated cells whose presence is necessary for cell proliferation. Among these conjugated, supramolecular structures have been developed with either Fab′ or scFv fragments. The preclinical trials performed show an effective degree of internalization in the cells that overexpress the HER2 receptors and an effective intracellular release of the drug.

Similarly it is possible to find in the literature various examples of functionalized liposomes with various types of peptide fragments such as VIP peptide (vasoactive intestinal) which recognize the overexpressed VIP receptors in tumour cells in the brain, and RGD peptide which selectively recognizes various types of α-integrins such as αᵥβ₃ and αᵥβ₅ which are both overexpressed in cases where there is a tumour.

The location of liposomal aggregates loaded with anti-tumour drugs such as doxorubicin can be followed in vivo through the introduction of a contrast medium for imaging techniques such as positron emission tomography (PET), computerized tomography (CT) and magnetic resonance imaging (MRI). There are also examples in the literature of liposomes for drug delivery and functionalized contrast media with peptides or antibodies for selective drug delivery into organs with tumours. In 2006, J. Gao (Nano Lett., 2006, 6 (11), pp 2427-2430) reported a liposomal formulation capable of co-encapsulating superparamagnetic iron nanoparticles (SPIO) and doxorubicin. The outer surface of the liposomes is functionalized with the peptide cRGD which selectively recognizes the receptors of the αᵥβ₃ integrins overexpressed in the tumour cell.

Finally, in WO2006/128643, A2 supramolecular aggregates are described which are formulated with functionalized amphiphilic molecules with a bioactive peptide for the selective targeting of overexpressed membrane receptors from tumour cells and/or with a chelating agent capable of complexing radioactive or paramagnetic metal ions for the real time in vivo display of the overexpressed targets and for studies of the biodistribution of the aggregate. These aggregates are claimed for their dual application of drug and contrast media delivery and are obtained by various approaches: co-aggregation of two amphiphilic monomers, one containing the chelating agent and one containing the peptide, or co-aggregation of a single monomer containing in the same molecule, as well as the two hydrophobic tails, both the peptide and the chelating agent with a commercial lipid; or from the co-aggregation of the two amphiphilic “gemini” type compounds, one containing two copies of the peptide and one containing two copies of the chelating agent. All these systems, subsequently identified using the term Naposomes, were found to be suitable for the targeting, both in vitro and in vivo, of tumour cells expressing receptors for the CCK8 peptide, octreotride, an analogue of somatostatin or for bombesin peptide.

It has now been established that within the classes of compounds described in WO2006/128643 A2, liposomal systems obtained from the co-aggregation of one or more commercial lipids with a single synthetic monomer simultaneously containing, in the same molecule: the peptide sequence of the bombesin peptide (or analogues thereof), the chelating agent, and two hydrophobic alkyl chains spaced by a polyoxyethyl (PEG) type linker from the chelating agent and from the peptide, are extremely effective due to the properties of: optimal ability to form liposomes of dimensions suitable for pharmaceutical use by intravenous administration, low degree of polydispersity, high stability of the liposomes over time, ability to encapsulate the cytotoxic drug doxorubicin in the most suitable quantities relative to the amphiphilic molecules used to formulate the aggregates, and good binding ability of the GRP receptors overexpressed by tumour cells. These systems therefore seem particularly suited to convey the drug doxorubicin in a selective way to tumour cells expressing GRP receptors, such as those in ovarian tumours.

### DETAILED DESCRIPTION OF THE INVENTION

The subject matter of the invention is defined by the appended claims.

The supramolecular aggregates, having in particular a liposomal structure, which are the subject of the present invention are made up of 1) one or more ionic surfactants normally used for the preparation of liposomes; and 2) a monomer having the general formula (I). The monomer of general formula (I) is preferably present in the final composition of the liposome in percentages variable between 0.5 and 15% in moles, more preferably between 1 and 5%. As well as ionic surfactants, cholesterol (to rigidify and stabilize the liposomal membrane) and sugars (for example trehalose) to preserve the integrity of the liposome during any lyophilization processes can be added to the final composition.

The surfactant present in the greatest quantity is a phospholipid, preferably a phosphatidylcholine, characterized by the presence of saturated or unsaturated fatty acids, or both; preferably the phospholipids can be selected from one of the following molecules or mixtures thereof: soy phosphatidylcholine (SPC), egg phosphatidylcholine (EPC), 1,2-dioleoyl-sn-glycero-3-phosphocholine (DOPC), 1,2-dipalmitoyl-sn-glycero-3-phosphocholine (DPPC), 1,2-disteroyl-sn-glycero-3-phosphocholine (DSPC), hydrogenated soy phosphatidylcholine (HSPC), hydrogenated egg phosphatidylcholine (HEPC) and phosphtidylglycerol (PG); other lipids can be combined with the phospholipid or phospholipids, for example cholesterol and derivatives thereof.

The monomer of general formula (I) is made up of three fundamental units, H, P and C, linked via linkers, L₁ and L₂, and a branched molecule Y. The general formula can be shown as:

H-L₁-Y(C)-L₂-P (1),

where:
H represents a hydrophobic group of formula (II), as defined in the appended claims.
C represents a chelating group selected from the group consisting of: a polyamino polycarboxylic acid residue and derivatives thereof, in particular selected from diethylenetriamino pentaacetic acid (DTPA), 1,4,7,10-tetraazacyclododecan-1,4,7,10-tetraacetic acid (DOTA), 1,4,7,10-tetraazacyclododecan-1,4,7-triacetic acid (DO3A), [10-(2-hydroxypropyl)-1,4,7,10-tetraazacyclododecan-1,4,7-triacetic acid (HPDO3A), 4-carboxy-5,8,11-tris(carboxymethyl)-1-phenyl-2-oxa-5,8,11-triazatridecan-3-oic acid (BOPTA), N-[2-[bis(carboxymethyl)amino]-3-(4-ethoxyphenyl)propyl]-N-[2-[bis(carboxymethyl)-amino]ethylglycine (EOB-DTPA), N,N-bis[2-[(carboxymethyl)[(methylcarbamoyl)-methyl]amino]ethyl]glycine (DTPA-BMA), 2-methyl-1,4,7,10-tetraazacyclododecan-1,4,7,10-tetraacetic acid (MCTA), (α,α′,α″,α′″)-tetramethyl-1,4,7,10-tetraazacyclododecan-1,4,7,10-tetraacetic acid (DOTMA), 1,4,7-triazacyclononane-N-glutaric N,N-diacetic acid (NODAGA), 1,4,7-triazacycloonane-N-succinic N,N-diacetic acid (NODASA), 1,4,7-triazacyclononanetriacetic acid (NOTA), ethylendiaminetetraacetic acid (EDTA), 1,4,7,10-tetraazacyclotridecan 1,4,7,10-tetraacetic acid (TRITA), 1,4,8,11-tetraazacyclotetradecan 1,4,8,11-tetraacetic acid (TETA), or it is a residue of a polyaminophosphate acid linker or derivatives thereof, in particular N,N′-bis-(pyridoxal-5-phosphate)ethylene-diamine-N,N′-diacetic acid (DPDP) and ethylenedinitrilotetrakis(methylphosphonic) acid (EDTP); or it is a residue of a polyaminophosphonic acid binder and derivatives thereof, or polyaminophosphonic acid and its derivatives, in particular 1,4,7,10-tetraazacyclododecan-1,4,7,10-tetrakis[methylene(methylphosphonic)] acid and 1,4,7,10-tetraazacyclododecan 1,4,7,10-tetrakis[methylene(methylphosphinic)] acid, or it is the residue of macrocyclic chelating agents such as texaphyrins, porphyrins and phthalocyanines; or it is DTPAGlu (N,N-Bis[2-[bis[2-(1,1-dimethylethoxy)-2-oxoethyl]-amino]ethyl]-L-glutamic acid 1-(1,1-dimethylethyl) ester or it is DTPALys (N,N-Bis[2-[bis[2-(1,1-dimethylethoxy)-2-oxoethyl]-amino]ethyl]-lysine 1-(1,1-dimethylethyl) ester.

DTPA, DTPAGlu or DOTA are particularly preferable.

The chelating group is linked to Y via a carboxyl group with formation of an amide bond.

Included within the scope of the invention are supramolecular aggregates wherein the chelating group C can form complexes with bivalent or trivalent ions of the elements having an atomic number varying among 20 and 31, 39, 42, 43, 44, 49, or between 57 and 83, radioactive isotopes of metals (^{99m}Tc, ²⁰³Pb, ⁶⁷Ga, ⁶⁸Ga, ⁷²As, ¹¹¹In, ¹¹³In, ⁹⁰Yt, ⁹⁷Ru, ^{82m}Rb, ⁶²Cu, ⁶⁴Cu, ⁵²Fe, ^{52m}Mn, ¹⁴⁰La, ¹⁷⁵Yb, ¹⁵³Sm, ¹⁶⁶Ho, ¹⁴⁹Pm, ¹⁷⁷Lu, ¹⁴²Pr, ¹⁵⁹Gd, ²¹²Bi, ⁴⁷Sc, ¹⁴⁹Pm, ⁶⁷Cu, ¹¹¹Ag, ¹⁹⁹Au, ¹⁸⁸Re, ¹⁸⁶Re, ¹⁶¹Tb and ⁵¹Cr) or paramagnetic metal ions Fe²⁺, Gd³⁺, Eu³⁺, Dy³⁺, La³⁺, Yb³⁺ or Mn²⁺ and compounds with radioactive isotopes of halogens (¹²³I, ¹²⁵I, ¹³¹I, ⁷⁵Br, ⁷⁶Br, ⁷⁴Br, ⁸²Br).

Particularly preferable are complexes with Fe²⁺, Gd³⁺, Eu³⁺, Dy³⁺, La³⁺, Yb³⁺, Mn²⁺, Fe³⁺, Cu²⁺, Cr³⁺, for display by MRI, or with radioisotopes such as ⁵¹Cr, ⁶⁷Ga, ⁶⁸Ga, ¹¹¹In, ^{99m}Tc, ¹⁴⁰La, ¹⁷⁵Yb, ¹⁵³Sm, ¹⁶⁶Ho, ⁹⁰Y, ¹⁴⁹Pm, ¹⁷⁷Lu, ⁴⁷Sc, ¹⁵⁹Gd, ²¹²Bi for display using the SPECT and PET techniques.
P is a peptide belonging to the class of peptides known as bombesin, hence the endogenous sequence of bombesin peptide.
The group P also includes:
C-terminal fragments of the bombesin peptide containing 6-9 amino acid residues;
analogues of the C-terminal fragment of the bombesin peptide having the general formula:

AA1-Gln-Trp-Ala-Val-AA2-His-AA3-AA4-NH₂

where:
AA1 is DPhe, D-Cpa, D-Tyr, D-Trp or is absent,

### AA2 is NMeGly, Gly or β-Ala,

### AA3 is Leu, Cha, Sta, Met or Nle.

### AA4 is Met, Leu or Nle.

All the peptides selected as the unit P of the molecule of formula (I) are capable of binding with nanomolar affinity to the receptors in class GRP, BB1, BB2, BB3, BB4 or other bombesin receptors and may or may not activate these receptors. The peptides are bound to L₂ via the N-terminal amino group of the amino acid sequence with formation of an amide bond. The preferred peptide sequences, containing natural or non natural amino acids, are shown in Table 1.

TABLE 1

| # | Preferred peptide sequences |
|---|---|
| 1) | -Gln-Trp-Ala-Val-Gly-His-Leu-Nle-NH₂ |
| 2) | -Gln-Trp-Ala-Val-Gly-His-Cha-Nle-NH₂ |
| 3) | -Gln-Trp-Ala-Val-Gly-His-Cha-Nle-Glu-NH₂ |
| 4) | -Gln-Trp-Ala-Val-Gly-His-Sta-Leu-NH₂ |
| 5) | -Gln-Trp-Ala-Val-NMeGly-His-Sta-Leu-NH₂ |
| 6) | -DPhe-Gln-Trp-Ala-Val-Gly-His-Sta-Leu-NH₂ |
| 7) | -DPhe-Gln-Trp-Ala-Val-Gly-His-Cha-Nle-NH₂ |
| 8) | -DPhe-Gln-Trp-Ala-Val-NMeG1y-His-Sta-Leu-NH₂ |
| 9) | -Gln-Trp-Ala-Val-Gly-His-Leu-Met-NH₂ |
| 10) | -DPhe-Gln-Trp-Ala-Val-NMeGIy-His-Leu-Nle-NH₂ |

Y is a branched molecule containing at least three reactive functions, preferably two amine functions and one carboxyl function so as to be able to bind, by the formation of amide bonds, L₁, C and L₂. The preferred molecules are natural amino acids such as lysine or non natural amino acids such as ornithine and 2,3-diaminopropionic acid (DAP). Alternatively, Y can be a branched molecule containing two carboxyl functions and one amine function such as for example glutamic acid or aspartic acid in order to form amide bonds with a chelating agent C having a free amine function.
L₁ is a spacer of polyoxyethylene type (Peg or analogues) or a sequence of molecules containing polyoxyethylene functions preferably sequentially linked via amide bonds, with total molecular weight between 1200 and 1800 Dalton, where L₁ contains at least one amine function and one carboxyl function necessary for the formation of amide bonds with H and Y, respectively.
L₂ is a spacer of polyoxyethylene type (Peg or analogues) or a sequence of molecules containing polyoxyethylene functions, preferably sequentially linked via amide bonds, with a total molecular weight between 200 and 800 Dalton, where L₂ contains at least one amine function and one carboxyl function necessary for the formation of amide bonds with Y and P, respectively.

It follows from the present invention that a spacer L₁ of greater length and therefore larger than L₂ is necessary in order to favour the aggregation and stability properties of the liposomes. In particular, in the development of the invention a comparative study was performed between various linkers with different hydrophilic, steric and pharmacokinetic properties, in order to suitably modulate the distance between the components of the hydrophobic molecule: peptide, chelating agent and hydrophobic chains.

Hence a spacer L₁ must have a molecular mass between 1200 and 1800 Dalton, namely masses corresponding to the length of the linker which make it possible to have the chelating group C and the peptide P sufficiently far from the liposome surface. Conversely, greater masses, corresponding to greater lengths, result in a rearrangement of the molecule, which can wrap the chelating agent in a pocket, not exposing the charges to the solvent medium, influencing the dimensions and shape.

Likewise the length of linker L₂ must necessarily be associated with a mass of between 200 and 800 Dalton.

Greater masses tend to obscure the peptide and render it unavailable for binding, thus lesser masses also prevent the correct interaction between the receptor and the amino acid sequence, for steric reasons.

These considerations, supported by experimental data (see examples 2f and 2g), involve an interference in the process of aggregation of the monomers. This innovative aspect makes it possible to obtain aggregates whose properties (shape, dimension and liposome stability) allow effective use in vivo and above all a reproducibility such that they can exceed the parameters required by the regulatory authorities with regard to what was previously verified and reported in WO 2006/128643 A2.

The compounds of formula (I) can be synthesized by known techniques, such as solid phase peptide synthesis, peptide synthesis in solution, synthetic methods of organic chemistry, or any combination of these. Synthetic methods based on suitable combinations of solid phase techniques and conventional methods in solution, which involve low production costs, in particular on an industrial scale, are preferably used.

In detail, such methods consist of the following steps: 1) solid phase synthesis of the peptide sequence, 2) introduction, still in solid phase, of the linker identified as L₂, 3) introduction of the molecule Y orthogonally protected on the two amine functions, 4) deprotection of one of the two amine functions, 5) introduction of the chelating system C, protected on the amine or carboxyl functions, 6) introduction, still in solid phase, of the linker identified as L₁ 7) introduction of the molecule H, and 8) detachment of the protected and purified peptide of the final compound from the resin using chromatographic techniques.

### EXAMPLES

### Example 1

### Synthesis of the Monomer of Formula (I)

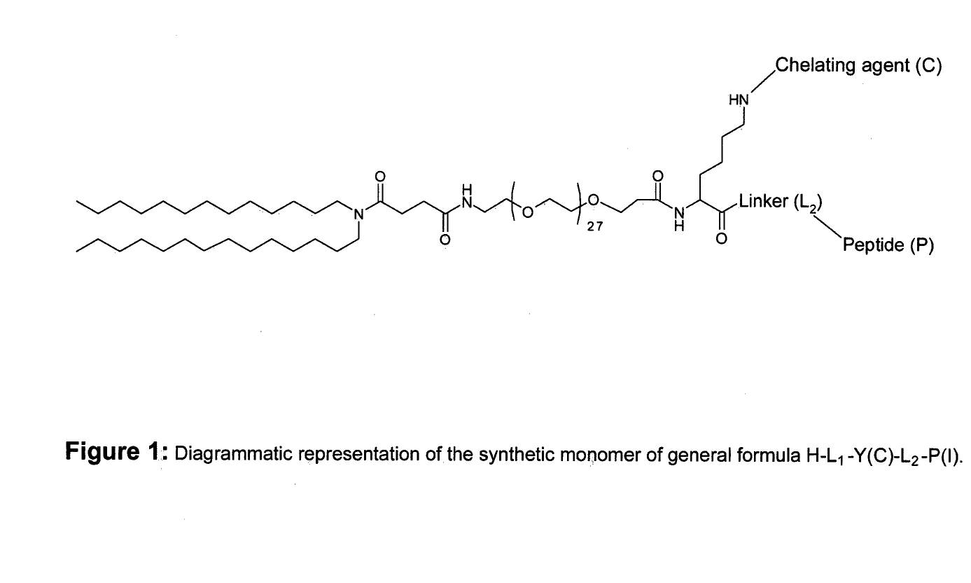

The monomer of formula (I) shown in FIG. 1 was synthesized using solid phase synthesis (SPPS) with Fmoc chemistry, growing the peptide on a polymeric type of support, as discussed in Chang, W. C. and White, P. D.; Fmoc solid phase peptide synthesis; Oxford Univ. Press (2000), New York. The monomer of general formula (I) shown in FIG. 1 was synthesized using solid phase synthesis. In, the present invention, the selected resin is the Rink-amide (0.78 mmol/g, 0.5 mmol scale, 0.64 g) which releases the peptide carboxamide at the C-terminal end. As a first step the Fmoc protective group is removed from the resin using a mixture of DMF/pip 70/30.

Next, the peptide fragment belonging to the class of peptides known as bombesin, C-terminal fragments containing 6-9 amino acid residues, analogues stabilized by the introduction of non natural amino acids, peptidomimetic analogues, analogues acting as agonists on the receptor and analogues acting as antagonists are synthesized. The peptide sequences synthesized are stated above. The synthesis of the peptide portion is performed using sequential couplings of the individual protected amino acids. All the couplings are repeated twice in DMF for 1 hour, using an excess of 4 equivalents for the individual amino acid derivative.

The α-amino acids are activated in situ by standard SPPS procedures which use HOBt/PyBop/DIPEA as activating agents. At the end of the coupling reaction, the Fmoc protective group in the main chain is removed by subjecting the resin to two deprotection cycles with DMF/pip 70/30 for 7 mins. After each Fmoc group deprotection step and after each amino acid condensation, the result is checked using a qualitative analytical test (ninhydrin test). At the end of the synthesis of the peptide fragment, the Fmoc protective group is removed from the final amino acid residue and the condensation is performed of L₂, or a polyoxyethylene type spacer (Peg or similar) of molecular weight between 200 and 800, which has either a carboxyl or an amine function. The carboxyl function is free and gives rise to the formation of the amide bond with the peptide growing on the resin, whilst the amine function is protected by the Fmoc group.

The L₂ spacer is condensed in a single coupling of 1 hour using two equivalents of L₂ relative to the scale of synthesis. After removal of the protective Fmoc group, the condensation of molecule Y is performed by double coupling with 4 equivalents under standard conditions. Y is a branched molecule (lysine, ornithine, Dap, aspartic acid or glutamic acid) containing two amine functions and one carboxyl function or else two carboxyl functions and one amino in order to be able to link, by formation of amide bonds, L₁, C and L₂. The Y molecule, as well as the protective Fmoc group in the main chain, also contains an orthogonal protective group in a side-chain, which can be removed under mild acidic or basic conditions.

After removal of the orthogonal group, by the standard procedure, the C group was condensed using an excess of 2 equivalents, as HATU activator, and 4 equivalents of DIPEA in DMF. C represents a chelating group (DTPA, DOTA, DO3A, HPDO3A, BOPTA, EOB-DTPA, DTPA-BMA, MCTA, DOTMA, DPDP, EDTP, DTPAGlu and DTPALys) protected on all the reactive (carboxyl) functions with the exception of that which has to react for the formation of the amide bond. The chelating agents DTPA, DTPAGlu or DOTA are particularly preferable.

At this point removal of the protective Fmoc group from the main chain of the lysine residue is effected, and also condensation of the ethoxyl spacer L₁. L₁ is a polyoxyethylene type spacer (Peg or similar) of molecular weight between 1200 and 1800. L₁ is condensed using an excess of two equivalents, as HATU activator, and 4 equivalents of DIPEA in DMF. After removal of the Fmoc protective group, a residue is condensed at the free terminal αNH₂ function of N,N-dioctadecylsuccinaminic acid using an excess of 4 equivalents and HBTU and HOBt as activators dissolved in a solution made up of equal parts of DCM/DMF/NMP.

After washing and drying the resin with DMF, DCM and ether, detachment of the peptide derivative from the resin was effected employing as cleavage mixture a solution of TFA/water/TIS in ratios 95.5/2.5/2.0, allowing the system to stand for 2 hours with stirring. The precipitation of the crude product takes place in ether/cold water in the usual way. The crude peptide is dissolved in water and acetonitrile and lyophilized.

The peptide derivative is purified by preparative chromatography (Method 3, reverse phase column Phenomenex C4; eluents H₂O 0.1% TFA and CH₃CN 0.1% TFA; elution gradient: from 5% B to 70% B in 10 minutes, from 70% B to 95% B in 10 minutes) and then characterized by LC/MS (Phenomenex C4 column).

TABLE 2
In the table the following are specified for each single example:
the peptide sequence P; the chelating agent C, the spacers L₁ and
L₂ and the retention times (Rₜ) and the mass of the compounds of
general formula H-L₁-Y(C)-L₂-P synthesized, purified and charac-
terized according to the standard procedure stated in example 1.

| Example | Peptide sequence | Chelating | Spacer | Spacer | Rt | MW |
|---|---|---|---|---|---|---|
| # | (P) | agent (C) | (L₁) | (L₂) | (min) | (Da) |
| 1.a | Gln-Trp-Ala-Val-Gly- | DTPA | H-Peg27-OH | H-AhOh-OH | 18.7 | 3664 |
| | His-Leu-Nle-NH₂ | | | | | |
| 1.b | DPhe-Gln-Trp-Ala- | DTPA | H-Peg27-OH | H-AhOh-OH | 18.5 | 3851 |
| | Val-Gly-His-Sta-Leu- | | | | | |
| | NH₂ | | | | | |
| 1.c | DPhe-Gln-Trp-Ala- | DTPA | H-Peg27-OH | H-AhOh-OH | 19.2 | 3849 |
| | Val-Gly-His-Cha-Nle- | | | | | |
| | NH₂ | | | | | |
| 1.d | DPhe-Gln-Trp-Ala- | DTPA | H-Peg27-OH | H-AhOh-OH | 19.0 | 3868 |
| | Val-NMeGly-His-Sta- | | | | | |
| | Leu-NH₂ | | | | | |
| 1.e | Gln-Trp-Ala-Val-Gly- | DTPA | H-Peg27-OH | H-AhOh-OH | 18.2 | 3682 |
| | His-Leu-Met-NH₂ | | | | | |
| 1.f | DPhe-Gln-Trp-Ala- | DTPA | H-Peg27-OH | H-AhOh-OH | 18.7 | 3825 |
| | Val-NMeGly-His-Leu- | | | | | |
| | Nle-NH₂ | | | | | |
| 1.g | Gln-Trp-Ala-Val-Gly- | DOTA | H-Peg27-OH | H-AhOh-OH | 19.5 | 3675 |
| | His-Leu-Nle-NH₂ | | | | | |
| 1.h | DPhe-Gln-Trp-Ala- | DOTA | H-Peg27-OH | H-AhOh-OH | 19.3 | 3862 |
| | Val-Gly-His-Sta-Leu- | | | | | |
| | NH₂ | | | | | |
| 1.i | DPhe-Gln-Trp-Ala- | DOTA | H-Peg27-OH | H-AhOh-OH | 20.1 | 3860 |
| | Val-Gly-His-Cha-Nle- | | | | | |
| | NH₂ | | | | | |
| 1.j | DPhe-Gln-Trp-Ala- | DOTA | H-Peg27-OH | H-AhOh-OH | 20.0 | 3879 |
| | Val-NMeGly-His-Sta- | | | | | |
| | Leu-NH₂ | | | | | |
| 1.k | Gln-Trp-Ala-Val-Gly- | DOTA | H-Peg27-OH | H-AhOh-OH | 19.1 | 3693 |
| | His-Leu-Met-NH₂ | | | | | |
| 1.l | DPhe-Gln-Trp-Ala- | DOTA | H-Peg27-OH | H-AhOh-OH | 19.6 | 3826 |
| | Val-NMeGly-His-Leu- | | | | | |
| | Nle-NH₂ | | | | | |
| 1.m | Gln-Trp-Ala-Val-Gly- | DTPA | H-Peg27-OH | H-dPeg(8)- | 18.4 | 3753 |
| | His-Leu-Nle-NH₂ | | | OH | | |
| 1.n | DPhe-Gln-Trp-Ala- | DTPA | H-Peg27-OH | H-dPeg(8)- | 18.7 | 3957 |
| | Val-NMeGly-His-Sta- | | | OH | | |
| | Leu-NH₂ | | | | | |
| 1.o | DPhe-Gln-Trp-Ala- | DTPA | H-Peg27-OH | H-Peg(12)- | 18.4 | 4425 |
| | Val-NMeGly-His-Leu- | | | OH | | |
| | Nle-NH₂ | | | | | |

### Example 1.a

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #1 stated in Table 1 (-Gln-Trp-Ala-Val-Gly-His-Leu-Nle-NH₂);
the chelating agent (C) is DTPA, introduced on the side-chain of the lysine residue, using the derivative protected on 4 of the 5 carboxylic groups using tert-butyl esters (DTPA(OtBu)₄-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 18.7 minutes and has an atomic mass equal to 3664 Da.

### Example 1.b

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #6 stated in Table 1 (-DPhe-Gln-Trp-Ala-Val-Gly-His-Sta-Leu-NH₂);
the chelating agent (C) is DTPA, introduced on the side-chain of the lysine residue, using the derivative protected on 4 of the 5 carboxylic groups using tert-butyl esters (DTPA(OtBu)₄-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 18.5 minutes and has an atomic mass equal to 3851 Da.

### Example 1.c

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #7 stated in Table 1 (-Dphe-Gln-Trp-Ala-Val-Gly-His-Cha-Nle-NH₂);
the chelating agent (C) is DTPA, introduced on the side-chain of the lysine residue, using the derivative protected on 4 of the 5 carboxylic groups using tert-butyl esters (DTPA(OtBu)₄-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 19.2 minutes and has an atomic mass equal to 3849 Da.

### Example 1.d

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #8 stated in Table 1 (-Dphe-Gln-Trp-Ala-Val-NMeGly-His-Sta-Leu-NH₂);
the chelating agent (C) is DTPA, introduced on the side-chain of the lysine residue, using the derivative protected on 4 of the 5 carboxylic groups using tert-butyl esters (DTPA(OtBu)₄-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 19.0 minutes and has an atomic mass equal to 3868 Da.

### Example 1.e

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #9 stated in Table 1 (-Gln-Trp-Ala-Val-Gly-His-Leu-Met-NH₂);
the chelating agent (C) is DTPA, introduced on the side-chain of the lysine residue, using the derivative protected on 4 of the 5 carboxylic groups using tert-butyl esters (DTPA(OtBu)₄-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 18.2 minutes and has an atomic mass equal to 3682 Da.

### Example 1.f

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #10 stated in Table 1 (-Dphe-Gln-Trp-Ala-Val-NMeGly-His-Leu-Nle-NH₂);
the chelating agent (C) is DTPA, introduced on the side-chain of the lysine residue, using the derivative protected on 4 of the 5 carboxylic groups using tert-butyl esters (DTPA(OtBu)₄-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 18.7 minutes and has an atomic mass equal to 3825 Da.

### Example 1.g

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #1 stated in Table 1 (-Gln-Trp-Ala-Val-Gly-His-Leu-Nle-NH₂);
the chelating agent (C) is the DOTA, introduced on the side-chain of the lysine residue, using the derivative protected on 3 of the 4 carboxylic groups using tert-butyl esters (DOTA(OtBu)₃-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 19.5 minutes and has an atomic mass equal to 3675 Da.

### Example 1.h

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #6 stated in Table 1 (-Dphe-Gln-Trp-Ala-Val-Gly-His-Sta-Leu-NH₂);
the chelating agent (C) is the DOTA, introduced on the side-chain of the lysine residue, using the derivative protected on 3 of the 4 carboxylic groups using tert-butyl esters (DOTA(OtBu)₃-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 19.3 minutes and has an atomic mass equal to 3862 Da.

### Example 1.i

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #7 stated in Table 1 (-Dphe-Gln-Trp-Ala-Val-Gly-His-Cha-Nle-NH₂);
the chelating agent (C) is the DOTA, introduced on the side-chain of the lysine residue, using the derivative protected on 3 of the 4 carboxylic groups using tert-butyl esters (DOTA(OtBu)₃-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 20.1 minutes and has an atomic mass equal to 3860 Da.

### Example 1.j

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #8 stated in Table 1 (-Dphe-Gln-Trp-Ala-Val-NMeGly-His-Sta-Leu-NH₂);
the chelating agent (C) is the DOTA, introduced on the side-chain of the lysine residue, using the derivative protected on 3 of the 4 carboxylic groups using tert-butyl esters (DOTA(OtBu)₃-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 20.0 minutes and has an atomic mass equal to 3879 Da.

### Example 1.k

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #9 stated in Table 1 (-Gln-Trp-Ala-Val-Gly-His-Leu-Met-NH₂);
the chelating agent (C) is the DOTA, introduced on the side-chain of the lysine residue, using the derivative protected on 3 of the 4 carboxylic groups using tert-butyl esters (DOTA(OtBu)₃-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 19.1 minutes and has an atomic mass equal to 3683 Da.

### Example 1.l

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #10 stated in Table 1 (-Dphe-Gln-Trp-Ala-Val-NMeGly-His-Leu-Nle-NH₂);
the chelating agent (C) is the DOTA, introduced on the side-chain of the lysine residue, using the derivative protected on 3 of the 4 carboxylic groups using tert-butyl esters (DOTA(OtBu)₃-OH);
the spacer L₁ is the H-Peg27-OH, introduced using the protected derivative Fmoc-Peg27-OH;
the spacer L₂ is H-AhOh-OH, i.e. a polyoxyethylene type spacer (of molecular weight of ca. 330 uma), which has either a carboxyl function or an amine function, and is introduced using the protected derivative Fmoc-AhOh-OH.

The purified product is eluted in RP-HPLC with a retention time Rt: 19.6 minutes and has an atomic mass equal to 3826 Da.

### Example 1.m

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #9 stated in Table 1 (-Gln-Trp-Ala-Val-NMeGly-His-Sta-Leu-NH₂);
the chelating agent (C) is DTPA, introduced on the side-chain of the lysine residue, using the derivative protected on 4 of the 5 carboxylic groups using tert-butyl esters (DTPA(OtBu)₄-OH);
the spacer L₁ is the H-Peg27-OH, introduced using the protected derivative Fmoc-Peg27-OH;
the spacer L₂ is H-dPeg(8)-OH, i.e. a polyoxyethylene type spacer (of molecular weight of ca. 425 uma), which has either a carboxyl function or an amine function, and is introduced using the protected derivative Fmoc-dPeg(8)-OH.

The purified product is eluted in RP-HPLC with a retention time Rt: 18.4 minutes and has an atomic mass equal to 3753 Da.

### Example 1.n

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #8 stated in Table 1 (-DPhe-Gln-Trp-Ala-Val-NMeGly-His-Sta-Leu-NH₂);
the chelating agent (C) is DTPA, introduced on the side-chain of the lysine residue, using the derivative protected on 4 of the 5 carboxylic groups using tert-butyl esters (DTPA(OtBu)₄-OH);
the spacer L₁ is the H-Peg27-OH, introduced using the protected derivative Fmoc-Peg27-OH;
the spacer L₂ is H-dPeg(8)-OH, i.e. a polyoxyethylene type spacer (of molecular weight of ca. 425 uma), which has either a carboxyl function or an amine function, and is introduced using the protected derivative Fmoc-dPeg(8)-OH.

The purified product is eluted in RP-HPLC with a retention time Rt: 18.7 minutes and has an atomic mass equal to 3957 Da.

### Example 1.o

The compound reported in the present example is the monomer of formula (I) shown in FIG. 1. It was synthesized, purified and characterized in accordance with the synthesis procedures stated in example 1. The peptide sequence P, the chelating agent C and the spacers L₁ and L₂ of the present compound are specified below:
the peptide sequence (P) synthesized in the present example is represented by the sequence #10 stated in Table 1 (-Dphe-Gln-Trp-Ala-Val-NMeGly-His-Leu-Nle-NH₂);
the chelating agent (C) is DTPA, introduced on the side-chain of the lysine residue, using the derivative protected on 4 of the 5 carboxylic groups using tert-butyl esters (DTPA(OtBu)₄-OH);
the spacer L₁ is the H-Peg27-OH, introduced using the protected derivative Fmoc-Peg27-OH;
the spacer L₂ is H-Peg(12)-OH, i.e. a polyoxyethylene type spacer (of molecular weight of ca. 600 uma), which has either a carboxyl function or an amine function, and is introduced using the protected derivative Fmoc-Peg(12)-OH.

The purified product is eluted in RP-HPLC with a retention time Rt: 18.4 minutes and has an atomic mass equal to 4425 Da.

### Example 2

### Formulation of the Liposomal Aggregates

The liposomes are prepared using the technique of evaporation of the lipid film. In brief, the lipid mixture made up of a phospholipid (DPPC, DSPC, or others) and a synthetic type compound of general formula (1), containing the peptide and the chelating agent, is dissolved in an organic mixture containing chloroform/methanol (2:1 v/v). The organic solution is transferred into a 50 mL glass flask and the solvent is evaporated in a rotavapor at reduced pressure in the presence of nitrogen. The resulting lipid film is hydrated with 1 ml of ammonium sulphate or ammonium citrate buffer (250 mM) of pH 5.5 or pH 4.0 respectively in the presence of glass beads for 2 hours at 65° C.

The resulting liposomal suspension is then extruded using an extruder, repeatedly passing the suspension through the polycarbonate membranes of pore diameter decreasing from 0.4 to 0.1 μm under a stream of nitrogen. The liposomal suspension is purified through Sephadex G-50 columns and eluted in deionized water to eliminate the external buffer. After extrusion and after column purification, the liposomal aggregates obtained are characterized by Photon Correlation Spectroscopy (PCS) to determine the value of the hydrodynamic radius (Rₕ) and the polydispersity index (PI). These measurements are also performed over time to check the stability of the liposomes in solution over time.

### Example 2.a

In the present example, the liposomes are formulated from DPPC (1·10⁻² M) and from one of the compounds of general formula H-L₁-Y(C)-L₂-P (0.1 mM) (corresponding to a phospholipids/monomer ratio of formula (1) 99/1), the synthesis whereof was described in examples 1.a-1.f. The compounds relating to examples 1.a-1.f, which differ only in the peptide sequence (see Table 2), do not display substantial structural changes (Rₕ and PI). In fact all the aggregates obtained after extrusion display an Rₕ equal to 143.0±40.1 nm and a PI equal to 0.150±0.04. These values remained unchanged after purification and over time.

### Example 2.b

In the present example, the liposomes are formulated from DPPC (1·10⁻²M) and from one of the compounds of general formula H-L₁-Y(C)-L₂-P (0.3 mM) (corresponding to a phospholipids/monomer ratio of formula (1) 97/3), the synthesis whereof was described in examples 1.a-1.f. The compounds relating to examples 1.a-1.f, which differ only in the peptide sequence (see Table 2), do not display substantial structural changes (Rₕ and PI). In fact all the aggregates obtained after extrusion display an Rₕ equal to 131.1±42.4 nm and a PI equal to 0.184±0.05. These values remained unchanged after purification and over time.

### Example 2.c

In the present example, the liposomes are formulated from DSPC (1·10⁻²M) and from one of the compounds of general formula H-L₁-Y(C)-L₂-P (0.2 mM) (corresponding to a phospholipids/monomer ratio of formula (1) 98/2), the synthesis whereof was described in examples 1.a-1.f. The compounds relating to examples 1.a-1.f, which differ only in the peptide sequence (see Table 2), do not display substantial structural changes (Rₕ and PI). In fact all the aggregates obtained after extrusion display an Rₕ equal to 122.5±20.4 nm and a PI equal to 0.093±0.06. After purification the Rₕ remained unchanged, while an increase is seen in the PI (0.151).

### Example 2.d

In the present example, the liposomes are formulated from DSPC (1·10⁻²M) and from the compound of general formula H-L₁-Y(C)-L₂-P (0.3 mM) (corresponding to a phospholipids/monomer ratio of formula (1) 97/3), the synthesis whereof was described in examples 1.a-1.f. The compounds relating to examples 1.a-1.f, which differ only in the peptide sequence (see Table 2), do not display substantial structural changes (Rₕ and PI). In fact all the aggregates obtained after extrusion display an Rₕ equal to 136.5±23.5 nm and a PI equal to 0.113±0.08. After purification the Rₕ remained unchanged, while an increase is seen in the PI (0.180).

### Example 2.e

In the present example, the liposomes are formulated from DPPC (1·10⁻² M) and from the compound of general formula H-L₁-Y(C)-L₂-P (0.1 mM) (corresponding to a phospholipids/monomer ratio of formula (1) 99/1), the synthesis whereof was described in examples 1.g-1.1. The compounds relating to examples 1.g-1.1, which differ only in the peptide sequence (see Table 2), do not display substantial structural changes (Rₕ and PI). In fact all the aggregates obtained after extrusion display an Rₕ equal to 141.4±38.4 nm and a PI equal to 0.165±0.05. These values remained unchanged after purification.

### Example 2.f (Comparative)

In the present example, the liposomes are formulated from DSPC (1·10⁻² M) and from the compound referred to as MonY in various concentrations (0.1 mM, 0.2 mM or 0.3 mM). The compound MonY is analogous to the compound of general formula (1) and contains: as the peptide portion P, the [7-14]BN fragment; as the chelating agent C, the DTPA; as the hydrophobic portion H, a carboxylated amide of general formula C1C2N—C(O)—X—C(O), in which C1 and C2 are saturated 18 carbon aliphatic amines. Also, X is an alkyl chain of general formula (CH₂)n with n=2 and spacers L₁ and L₂ are polyoxyethylene type fragments (Peg) of molecular weight of 144 and 288 Dalton, respectively.

This is therefore not included in the description of the compound of general formula (1) since L₁<L₂ and L₁ does not come within the molecular weight range of between 1200 and 1800 Dalton. All the aggregates obtained (at the various molar ratios DSPC/MonY) display an Rₕ equal to 210.2±71.5 nm and a PI equal to 0.85±0.1. Due to an excessive PI, these liposomal formulations containing MonY in which L₁<L₂ cannot be suitable for use in vivo. In particular, it is considered that with a spacer L₁ of reduced dimensions the chelating agent DTPA and the peptide are too close to the surface of the liposome and influence the aggregation process, which gives rise to liposomes with high polydispersity and lower stability, unacceptable for use in pharmaceutical preparations.

### Example 2.g (Comparative)

In the present example, the liposomes are formulated from DSPC (1·10⁻² M) and from the compound referred to as MonY in various concentrations (0.1 mM, 0.2 mM or 0.3 mM). The compound MonY is analogous to the compound of general formula (1) and contains: as the peptide portion P, the [7-14]BN fragment; as the chelating agent C, the DTPA; as the hydrophobic portion H, a carboxylated amide of general formula C1C2N—C(O)—X—C(O), in which C1 and C2 are saturated 18 carbon aliphatic amines. Also, X is an alkyl chain of general formula (CH₂)n with n=2, and spacers L₁ and L₂ are polyoxyethylene type fragments (Peg) both of molecular weight of 1500 Dalton.

This is therefore not included in the description of the compound of general formula (1) since L₁=L₂ and L₂ does not come within the molecular weight range of between 200 and 800 Dalton. All the aggregates obtained (at the various molar ratios DSPC/MonY) display an Rₕ equal to 240.0±85.0 nm and a PI equal to 0.70±0.2. Due to an excessive PI, these liposomal formulations containing MonY in which L₁=L₂ cannot be suitable for use in vivo. In particular, it is considered that with an L₂ spacer of excessively large dimensions the peptide is unavailable for the binding, as it is covered and unacceptable for use in pharmaceutical preparations in which the peptide should be available for specific interaction with the receptors.

### Example 3

### Preparation of Liposomal Aggregates Containing Doxorubicin (DOX) and their Characteristics

An aqueous solution of cryoprotectant (trehalose, sucrose or lactose) was added to the liposomal suspensions described in examples 2.a-2.f. The liposomes were frozen in liquid nitrogen and lyophilized for 24 hours. The lyophilized formulation was then kept at −20° C. Each formulation was prepared in triplicate. The liposomal aggregates obtained were then taken up into solution again and mixed with a buffer solution containing DOX.

In brief, the buffer solution of HEPES of pH 7.4 is added to the lyophilized powder containing the liposomal aggregate (Reactant A) and to the DOX (Reactant B). The reactant A and the reactant B are mixed and incubated at 60° C. for 30 mins (ratio in grams of doxorubicin/lipids=0.100).

The average diameter of the liposomes, before and after lyophilization, was determined using Photon Correlation Spectroscopy (PCS). Each sample was diluted with deionized water and filtered and analysed at 20° C. For each sample, the average diameter and the size distribution were obtained from the average of three measurements. For each formulation, the average diameter and the polydispersity index (P.I.) were calculated from the average of three different batches.

The quantity of PC in the lyophilized formulations was determined by the Stewart test (Stewart JC. Anal Biochem. 1980). In brief, 100 μl of liposomal suspension was diluted with 400 μl of water containing ammonium ferrothiocyanate (0.1 N); the solution was then added to 500 μl of chloroform. The concentration of phospholipids was determined by measuring the absorbance of the organic phase at 485 nm.

The quantity of doxorubicin not encapsulated in the liposomal aggregates was determined as follows: 1 ml of liposomes containing DOX was ultracentrifuged (Optima Max E, Beckman Coulter, USA) at 80,000 rpm, at 4° C. for 40 mins. The supernatants were accurately removed and the concentration of DOX was determined by UV/Vis spectro-photometry at a wavelength of 480 nm. The results were expressed as encapsulation efficiency, calculated as the ratio between the quantity of DOX present in the supernatants and the quantity of DOX theoretically loaded.

### Example 4

### Labelling and In Vitro Testing of Binding on Cells Expressing GRP Receptors

The cell binding experiments were performed on target-selective liposomes DPPC/H-L₁-Y(C)-L₂-P (97/3) and DSPC/H-L₁-Y(C)-L₂-P (97/3) labelled with isotope 111 of radioactive indium. Experiments with non target-selective liposomes DPPC/(C18)₂DTPA (99.9/0.1) and DSPC/(C18)₂DTPA (99.9/0.1) and liposomes in which the peptide P, analogue of bombesin, was replaced by a peptide with scramble sequence (Ps) DPPC/H-L₁-Y(C)-L₂-P_{S} (97/3) and DSPC/H-L₁-Y(C)-L₂-P_{S}(97/3), were used as negative controls.

The labelling of the supramolecular aggregates was performed to a final concentration of 2*10⁴M. Traces of ¹¹¹InCl₃ were added to 1 mL of liposomes and the solution was brought up to a final volume of 2 mL by adding sodium acetate buffer (0.4 M, pH 5.0). The mixture was incubated for 30 mins at 90° C. The completeness and the efficiency of the reaction were checked using gel-filtration on a pre-packed Sephadex G-50 column (Pharmacia Biotech). The binding experiments were performed on PC-3 cells overexpressing the GRP receptor. The cells were inoculated on the previous day on 6-well plates at a density of 800,000-1,000,000 cells per well.

On the day of the experiment, the cell medium is removed, the cells are washed two times with fresh medium (DMEM with 1% of foetal bovine serum, pH 7.4) and incubated for at least one hour at 37° C. The cells were incubated (in triplicate) with 100 μL of liposomes labelled with indium 111/natural indium at various times (30 mins, 1 hour, 2 hours, 4 hours). The final volume is 1 mL and the final liposomal concentration is 2*10⁻⁴M. At the end of the incubation, the cell medium, containing the unbound radioactivity, is separated from the cells.

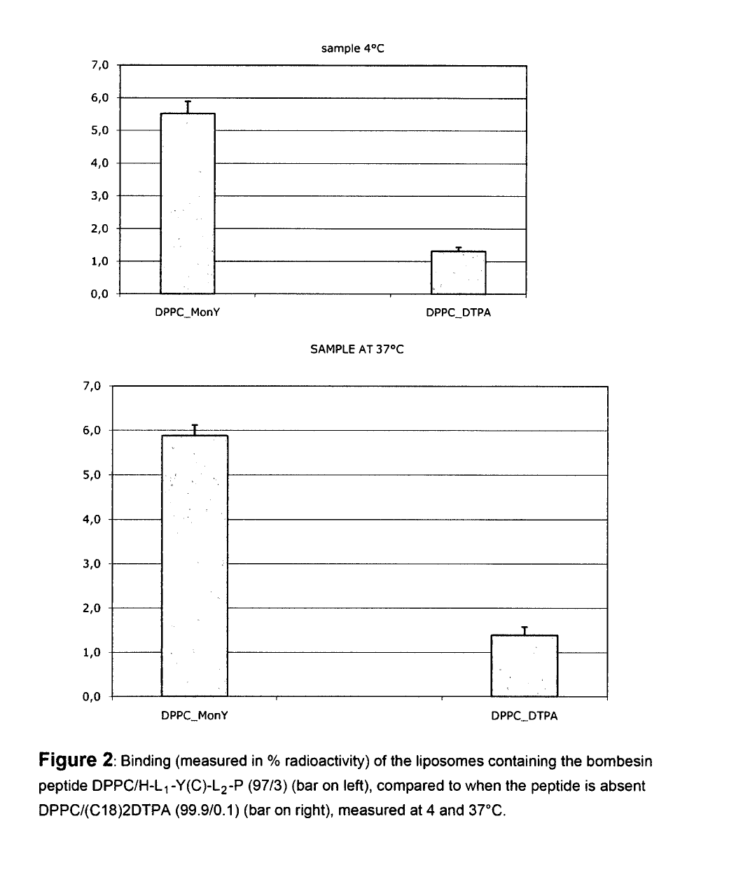

The radioactivity bound to the cells was recovered by tryptic digestion after two rapid washes with frozen PBS followed by 1 hour of incubation and measured by gamma counting. The difference in binding of the liposomes containing H-L₁-Y(C)-L₂-P with the bombesin peptide, compared to when the peptide is absent, is clearly visible in FIG. 2. Experiments were conducted with various type (1) monomers in the liposomal formulation, varying the peptide sequences and the chelating agent, such as those described in examples 1a-1o. No substantial differences are found in the target selective binding properties. For example, in FIG. 2 the result for the liposomes containing monomer 1a is reported.

### Example 5

### In Vitro Cytotoxicity Tests on Cells Expressing GRP Receptors

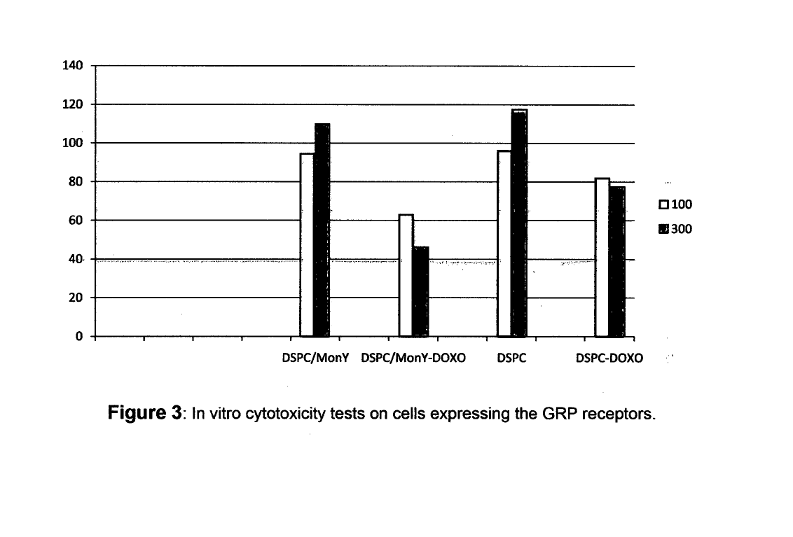

The cytotoxicity experiments were performed on PC-3 cells overexpressing the GRP receptor, using the MTT test. The cells were inoculated onto 96-well plates and incubated overnight so as to obtain their adhesion to the plate. On the following day, the culture medium is removed and the cells are incubated with a liposomal suspension with two concentrations of doxorubicin (100 ng/mL and 300 ng/mL). The systems studied are the liposomal compound of formula DSPC/H-L₁-Y(C)-L₂-P (97/3) loaded with doxorubicin, as stated in example 3, (abbreviated in FIG. 3 as DSPC/MonY-DOXO) in which the monomer H-L₁-Y(C)-L₂-P is the compound stated in example 1a, and the liposomal compound made up of DSPC alone loaded with doxorubicin (abbreviated in FIG. 3 as DSPC-DOXO). For comparison, the analogous liposomal systems were also studied without doxorubicin (abbreviated in FIG. 3 as DSPC/MonY and DSPC).

As shown in FIG. 3, incubation of the cells with the liposomal solution of DSPC/H-L₁-Y(C)-L₂-P (97/3) loaded with doxorubicin, shows a significantly lower cell survival compared to the incubation with the liposomal solution made up of DSPC alone loaded with equivalent quantities of doxorubicin, indicating selective binding externally of the functionalized liposomes with the bombesin peptide of sequence: Gln-Trp-Ala-Val-Gly-His-Leu-Nle-NH₂.

### Example 6

### In Vivo Activity Tests

In vivo experiments were performed on six-week old female BALB/c nude mice. The volume of the tumour induced by the inoculation of PC-3 cells was evaluated every two days from 28 days after inoculation of the cells. The treatment with the liposomal system was performed 36 days after the inoculation of the cells.

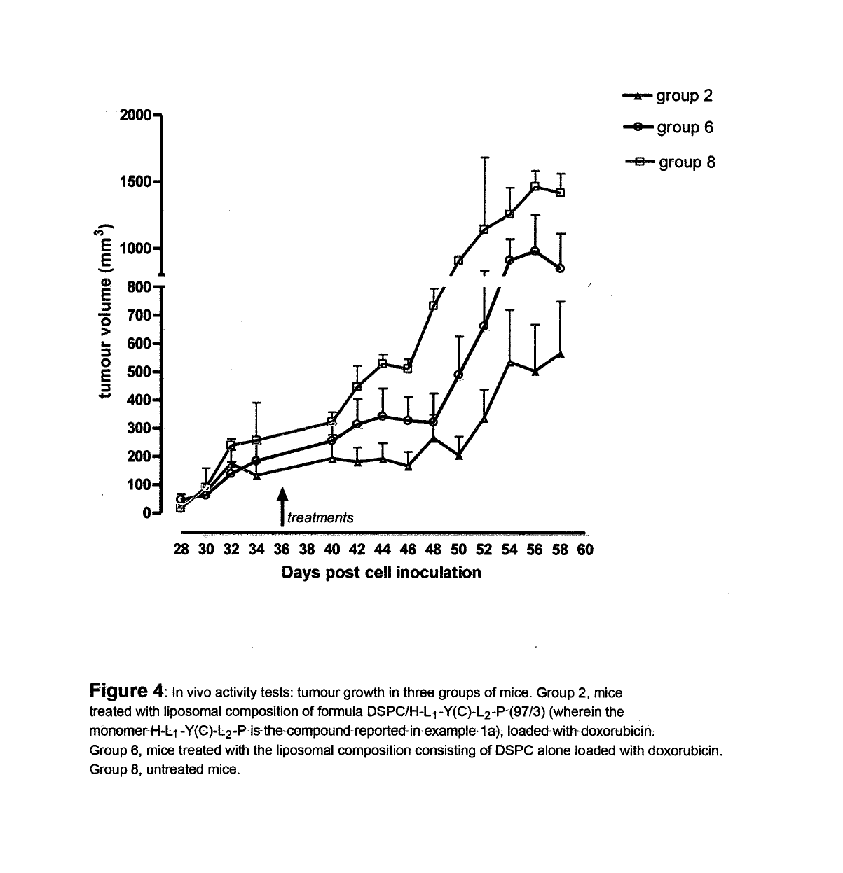

A group of 6 mice was treated with liposomal compound of formula DSPC/H-L₁-Y(C)-L₂-P (97/3) loaded with doxorubicin wherein the monomer H-L₁-Y(C)-L₂-P is the compound stated in example 1a. A second group of six mice was treated with the liposomal compound made up of DSPC alone loaded with doxorubicin. The quantity of liposomal suspension injected (100 microlitres) corresponds to a dose of doxorubicin of 10 mg/kg of the weight of the mouse. A third group of mice was treated with saline solution alone. The results, stated in FIG. 4 (average of six mice±s.e.), indicate that the growth of the tumour in the mice is greatly slowed using the liposomal system DSPC/MonY-DOXO compared to the mice treated with the liposomal system without peptide, DSPC-DOXO, and compared to the untreated mice.

1. A supramolecular aggregate with liposomal structure, the supramolecular aggregate comprising:
a) one or more ionic surfactants with phospholipid structure
b) an amphiphilic molecule of general formula (I)
H-L₁-Y(C)-L₂-P (I),
wherein:
H is a molecule with a carboxylic amide structure of general formula 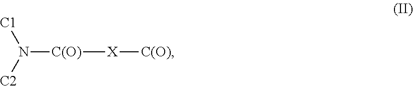
where
C1 and C2 are saturated, unsaturated or polyunsaturated hydrophobic, aliphatic hydrocarbon chains, equal (C1=C2) or different from each other (C1≠2) with a number of carbon atoms between 8 and 24 and X is an alkylene chain of general formula (CH₂)n with 1<n<6,
C is a chelating group selected from the group consisting of:
a polyamino polycarboxylic acid residue;
a residue of a polyamino phosphate acid linker;
a residue of a polyamino phosphonic acid linker or polyamino phosphinic acid linker;
a residue of macrocyclic chelating agents selected from texaphyrin, porphyrins and phthalocyanines;
DTPAGlu; and
DTPALys;
the chelating group being linked to Y through a carboxyl group with formation of an amide bond;
Y is a branched molecule containing at least three reactive functions so as to be capable of binding L₁, C and L₂;
P is a peptide selected from:
an endogenous bombesin peptide with SEQ ID NO: 1;
a C-terminal fragment of the bombesin peptide, the C-terminal fragment containing 6-9 amino acid residues;
an analogue of the C-terminal fragment of the bombesin peptide having general formula:
(SEQ ID NO: 2)
AA1-Gln-Trp-Ala-Val-AA2-His-AA3-AA4-NH₂,
where
AA1 is DPhe, D-Tyr, D-Trp or is absent;
AA2 is NMeGly, Gly or β-Ala;
AA3 is Leu, Cha, Sta, Met or Nle;
AA4 is Met, Leu or Nle;
L₁ is a spacer of polyoxyethylene type containing at least one amine function and a carboxyl function necessary for formation of amide bonds with H and Y, respectively, or a sequence of molecules containing polyoxyethylene functions, sequentially linked by amide bonds, said spacer having a total molecular weight between 1200 and 1800 Dalton;
L₂ is a spacer of polyoxyethylene type containing at least one amine function and one carboxyl function necessary to form amide bonds with Y and P, respectively, or a sequence of molecules containing polyoxyethylene functions, sequentially linked by amide bonds, said spacer having a total molecular weight between 200 and 800 Daltons.

2. The supramolecular aggregate according to claim 1, wherein C is a residue of a polyamino carboxylic acid selected from diethylenetriamine pentaacetic acid (DTPA), 1,4,7,10-tetraazacyclododecane-1,4,7,10-tetraacetic acid (DOTA), 1,4,7,10-tetraazacyclododecaen-1,4,7-triacetic acid (DO3A), [10-(2-hydroxypropyl)-1,4,7,10-tetraazacyclododecane-1,4,7-triacetic acid (HPDO3A), 4-carboxy-5,8,11-tris(carboxymethyl)-1-phenyl-2-oxa-5,8,11-triazatridecane-13-oic acid (BOPTA), N-[2-[bis(carboxymethyl)amino]-3-(4-ethoxyphenyl)propyl]-N-[2-[bis(carboxymethyl)amino]-ethylglycine (EOB-DTPA), N,N-bis[2-[(carboxymethyl)[(methylcarbamoyl)methyl]amino]ethyl]glycine (DTPA-BMA), 2-methyl-1,4,7,10-tetraazacyclo-dodecane-1,4,7,10-tetraacetic acid (MCTA), (α,α′,α″,α′″)-tetramethyl-1,4,7,10-tetraazacyclo-dodecane-1,4,7,10-tetraacetic acid (DOTMA), 1,4,7-triazacyclononane-N-glutaric N,N-diacetic acid (NODAGA), 1,4,7-triazacyclononane-N-succinic-N,N-diacetic acid (NODASA), 1,4,7-triazacyclononanetriacetic acid (NOTA), ethylendiaminetetraacetic acid (EDTA), 1,4,7,10-tetraazacyclotridecane-1,4,7,10-tetraacetic acid (TRITA) and 1,4,8,11-tetraazacyclotetradecane 1,4,8,11-tetraacetic acid (TETA).

3. The supramolecular aggregate according to claim 1, wherein C is a residue of a polyaminophosphate acid linker, selected from the group consisting of N,N′-bis-(pyridoxal-5-phosphate)ethylenediamine-N,N′-diacetic acid (DPDP) and ethylenedinitrilotetrakis(methylphosphonic) acid (EDTP).

4. The supramolecular aggregate according to claim 1, wherein C is selected from the group consisting of 1,4,7,10-tetraazacyclododecane-1,4,7,10-tetrakis[methylene(methylphosphonic)] acid and 1,4,7,10-tetraazacyclododecane-1,4,7,10-tetrakis[methylene(methylphosphinic)] acid.

5. The supramolecular aggregate according to claim 1, wherein C is selected from DTPA, DTPAGlu and DOTA.

6. The supramolecular aggregate according to claim 1, wherein P is a peptide selected from the group consisting of -Gln-Trp-Ala-Val-Gly-His-Leu-Nle-NH₂ (SEQ ID NO:3), -Gln-Trp-Ala-Val-Gly-His-Cha-Nle-NH₂ (SEQ ID NO:4), -Gln-Trp-Ala-Val-Gly-His-Cha-Nle-Glu-NH₂ (SEQ ID NO:5), -Gln-Trp-Ala-Val-Gly-His-Sta-Leu-NH₂ (SEQ ID NO:6), -Gln-Trp-Ala-Val-NMeGly-His-Sta-Leu-NH₂ (SEQ ID NO:7), -DPhe-Gln-Trp-Ala-Val-Gly-His-Sta-Leu-NH₂ (SEQ ID NO:8), -DPhe-Gln-Trp-Ala-Val-Gly-His-Cha-Nle-NH₂ (SEQ ID NO:9), -DPhe-Gln-Trp-Ala-Val-NMeGly-His-Sta-Leu-NH₂ (SEQ ID NO:10), -Gln-Trp-Ala-Val-Gly-His-Leu-Met-NH₂ (SEQ ID NO:11) and -Dphe-Gln-Trp-Ala-Val-NMeGly-His-Leu-Nle-NH₂ (SEQ ID NO:12).

7. The supramolecular aggregate according to claim 1, wherein Y is a natural amino acid, selected from the group consisting of lysine, glutamic acid and aspartic acid or a non natural amino acid, selected from the group consisting of ornithine and 2,3-diaminopropionic acid (DAP).

8. The supramolecular aggregate according to claim 1, wherein said ionic surfactant with phospholipid structure is selected from the group consisting of soy phosphatidylcholine (SPC), egg phosphatidylcholine (EPC) 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), hydrogenated soy phosphatidylcholine (HSPC), hydrogenated egg phosphatidylcholine (HEPC) and phosphatidylglycerol (PG).

9. The supramolecular aggregate according claim 1, wherein the amphiphilic molecule of general formula (I) is present in a final supramolecular aggregate composition in a percentage of 0.5% to 15% in moles.

10. The supramolecular aggregate according to claim 1, wherein the chelating group C is complexed with bivalent or trivalent ions of an element having an atomic number varying between 20 and 31, 39, 42, 43, 44, 49, or between 57 and 83, of a radioactive isotope of a metal selected from ^{99m}Tc, ²⁰³Pb, ⁶⁷Ga, ⁶⁸Ga, ⁷²As, ¹¹¹In, ¹¹³In, ⁹⁰Yt, ⁹⁷Ru, ^{82m}Rb, ⁶²Cu, ⁶⁴Cu, ⁵²Fe, ^{52m}Mn, ¹⁴⁰La, ¹⁷⁵Yb, ¹⁵³Sm, ¹⁶⁶Ho, ¹⁴⁹Pm, ¹⁷⁷Lu, ¹⁴²Pr, ¹⁵⁹Gd, ²¹²Bi, ⁴⁷Sc, ¹⁴⁹Pm, ⁶⁷Cu, ¹¹¹Ag, ¹⁹⁹Au, ¹⁸⁸Re, ¹⁸⁶Re, ¹⁶¹Tb and ⁵¹Cr or with paramagnetic metal ions Fe²⁺, Gd³⁺, Eu³⁺, Dy³⁺, La³⁺, Yb³⁺ or Mn²⁺ and a compound with radioactive isotopes of halogens selected from ¹²³I, ¹²⁵I, ¹³¹I, ⁷⁵Br, ⁷⁶Br, ⁷⁴Br, ⁷⁷Br, ⁸²Br.

11. The supramolecular aggregate according to claim 10, in form of complex with Fe²⁺, Gd³⁺, Eu³⁺, Dy³⁺, La³⁺, Yb³⁺, Mn²⁺, Fe³⁺, Cu²⁺ and Cr³⁺, or with radioisotopes such as ⁵¹Cr, ⁶⁷Ga, ⁶⁸Ga, ¹¹¹In, ^{99m}Tc, ¹⁴⁰La, ¹⁷⁵Yb, ¹⁵³Sm, ¹⁶⁶Ho, ⁹⁰Y, ¹⁴⁹Pm, ¹⁷⁷Lu, ⁴⁷Sc, ¹⁴²Pr, ¹⁵⁹Gd and ²¹²Bi.

12. An anti-tumour drug formulation comprising anti-tumour drug anchored to the supramolecular aggregate according to claim 1.

13. The anti-tumour drug formulation according to claim 12, wherein the anti-tumour drug is the drug doxorubicin.

14. The supramolecular aggregate according to claim 1, wherein C1 and C2 are hydrocarbon chains with a number of carbon atoms between 12 and 18.

15. The supramolecular aggregate according to claim 1, wherein Y is a branched molecule capable of binding L₁, C and L₂, by formation of amide bonds.

16. The supramolecular aggregate according claim 1, wherein the amphiphilic molecule of general formula (I) is present in-a final supramolecular aggregate composition in a percentage of 1% to 5% in moles.

17. A method of treating an individual with an anti-tumour drug, the method comprising
administering to the individual the anti-tumour drug anchored to the supramolecular aggregate of claim 1.

18. The method of claim 17, wherein the anti-tumour drug is doxorubicin

19. The method of claim 18, wherein the administering is performed to selectively delivering the doxorubicin to tumour cells expressing a GRP receptor.
